(19) United States
(12) Patent Application Publication
(10) Pub. No.: US 2026/0094267 A1
(43) Pub. Date: Apr. 2, 2026

(54) MULTI-MODAL MACHINE LEARNING TECHNIQUES FOR DETERMINING EMBRYONIC VIABILITY IN CLINICAL IN-VITRO FERTILIZATION (IVF)

(71) Applicant: President and Fellows of Harvard College, Cambridge, MA (US)

(72) Inventors: Hanspeter Pfister, Cambridge, MA (US); Junsik Kim, Cambridge, MA (US)

(73) Assignee: President and Fellows of Harvard College, Cambridge, MA (US)

(21) Appl. No.: 19/338,895

(22) Filed: Sep. 24, 2025

Related U.S. Application Data
(60) Provisional application No. 63/700,510, filed on Sep. 27, 2024.

(51) Int. Cl. G06T 7/00; G06T 7/11; G16H 10/60; G16H 50/20

(52) U.S. Cl. CPC G06T 7/0012; G06T 7/11; G16H 10/60; G16H 50/20; G06T 2207/30044

(57) ABSTRACT

Some aspects provide for techniques for selecting at least one embryo for transfer to a subject in furtherance of an in vitro fertilization (IVF) treatment. In some embodiments, the techniques comprise: obtaining video data for a plurality of embryos including a first embryo, the video data comprising a first sequence of image frames depicting the first embryo; obtaining electronic health data for the subject; predicting, using the video data and the electronic health data, respective degrees of viability of at least some of the plurality of embryos, the predicting comprising: processing the electronic health data and the first sequence of image frames using at least one trained machine learning model to obtain a first degree of viability of the first embryo; and selecting, from among the at least some of the plurality of embryos and based on the predicted degrees of viability, the at least one embryo for transfer.

### CROSS REFERENCE TO RELATED APPLICATIONS

The present application claims the benefit of and priority under 35 U.S.C. § 119(e) to U.S. Provisional Patent Application Ser. No. 63/700,510, entitled “MULTI-MODAL MACHINE LEARNING TECHNIQUES FOR DETERMINING EMBRYONIC VIABILITY IN CLINICAL IN-VITRO FERTILIZATION (IVF),” filed Sep. 27, 2024, which is herein incorporated by reference in its entirety.

### STATEMENT REGARDING FEDERALLY SPONSORED RESEARCH OR DEVELOPMENT

This invention was made with government support under HD104969 awarded by National Institutes of Health (NIH). The government has certain rights in the invention.

### BACKGROUND

Infertility affects approximately one in six couples globally, propelling many towards assisted reproductive technologies such as In-Vitro Fertilization (IVF). IVF entails stimulating patients to produce multiple oocytes, which are then retrieved, fertilized in vitro, and the resultant embryos cultured. Selected embryos are transferred to the maternal uterus to initiate pregnancy, with surplus viable embryos cryopreserved for future attempts.

### SUMMARY

Some aspects provide for a method for selecting at least one embryo for transfer to a subject in furtherance of an in vitro fertilization (IVF) treatment, the method comprising: using at least one processor to perform: obtaining video data for a plurality of embryos including a first embryo, the video data comprising a first sequence of image frames depicting the first embryo; obtaining electronic health data for the subject, the electronic health data comprising information about the IVF treatment; predicting, using the video data and the electronic health data, respective degrees of viability of at least some of the plurality of embryos, the predicting comprising: processing the electronic health data and the first sequence of image frames using at least one trained machine learning model to obtain a first degree of viability of the first embryo; and selecting, from among the at least some of the plurality of embryos and based on the predicted degrees of viability including the first degree of viability, the at least one embryo for transfer to the subject.

Some aspects provide for a system, comprising: at least one processor; and at least one non-transitory computer-readable storage medium storing processor-executable instructions that, when executed by the at least one processor, cause the at least one processor to perform a method for selecting at least one embryo for transfer to a subject in furtherance of an in vitro fertilization (IVF) treatment, the method comprising: obtaining video data for a plurality of embryos including a first embryo, the video data comprising a first sequence of image frames depicting the first embryo; obtaining electronic health data for the subject, the electronic health data comprising information about the IVF treatment; predicting, using the video data and the electronic health data, respective degrees of viability of at least some of the plurality of embryos, the predicting comprising: processing the electronic health data and the first sequence of image frames using at least one trained machine learning model to obtain a first degree of viability of the first embryo; and selecting, from among the at least some of the plurality of embryos and based on the predicted degrees of viability including the first degree of viability, the at least one embryo for transfer to the subject.

Some aspects provide for at least one non-transitory computer-readable storage medium storing processor-executable instruction that, when executed by at least one processor, cause the at least one processor to perform a method for selecting at least one embryo for transfer to a subject in furtherance of an in vitro fertilization (IVF) treatment, the method comprising: obtaining video data for a plurality of embryos including a first embryo, the video data comprising a first sequence of image frames depicting the first embryo; obtaining electronic health data for the subject, the electronic health data comprising information about the IVF treatment; predicting, using the video data and the electronic health data, respective degrees of viability of at least some of the plurality of embryos, the predicting comprising: processing the electronic health data and the first sequence of image frames using at least one trained machine learning model to obtain a first degree of viability of the first embryo; and selecting, from among the at least some of the plurality of embryos and based on the predicted degrees of viability including the first degree of viability, the at least one embryo for transfer to the subject.

Embodiments of any of the above aspects may have one or more of the following features.

Some embodiments further comprise: after selecting the at least one embryo for transfer, transferring the at least one embryo to the subject.

Some embodiments further comprise: after selecting the at least one embryo for transfer, generating a recommendation to transfer the at least one embryo to the subject; and providing an indication of the recommendation to a user.

In some embodiments, the information about the IVF treatment comprises an indication of a fertilization type and/or an indication of a number of oocytes retrieved from the subject.

In some embodiments, the electronic health data further comprises an indication of one or more measurements of the subject, the one or more measurements comprising measurements of one or more hormone levels of the subject, a weight of the subject, a height of the subject, a body mass index (BMI) of the subject, and/or an age of the subject.

In some embodiments, the electronic health data further comprises information about a medical history of the subject.

In some embodiments, the information about the subject's medical history comprises an indication of an age at which the subject first menstruated.

Some embodiments further comprise: generating, using the video data, morphological features for the at least some of the plurality of embryos. In some embodiments, predicting the respective degrees of viability of the at least some of the plurality of embryos comprises predicting the respective degrees of viability based on the electronic health data, the video data, and the morphological features.

In some embodiments, the morphological features comprise one or more morphological features for the first embryo, and predicting the first degree of viability of the first embryo comprises: processing the electronic health data, the first sequence of image frames, and the one or more morphological features using the at least one trained machine learning model to obtain the first degree of viability of the first embryo.

In some embodiments, the morphological features comprise, for each of the at least some of the plurality of embryos, a segmentation of a zona pellucida, a grading of a degree of fragmentation, a classification of a developmental stage, an object instance segmentation of cells in a cleavage stage, and/or an object instance segmentation of pronuclei before a first cell division.

Some embodiments further comprise: obtaining interpretable features for the at least some of the plurality of embryos. In some embodiments, predicting the respective degrees of viability of the at least some of the plurality of embryos comprises predicting the respective degrees of viability based on the electronic health data, the video data, and the interpretable features.

In some embodiments, the interpretable features comprise one or more interpretable features for the first embryo, and predicting the first degree of viability of the first embryo comprises: processing the electronic health data, the first sequence of image frames, and the one or more interpretable features using the at least one trained machine learning model to obtain the first degree of viability of the first embryo.

In some embodiments, the interpretable features comprise, for each of the at least some of the plurality of embryos, a zona pellucida thickness, a standard deviation of the zona pellucida thickness, one or more diameters of an inner zona pellucida region, one or more diameters of an outer zona pellucida region, one or more transition times between embryo development stages, one or more fragmentation levels, a zygote size, a zygote shape, one or more cell symmetry indices, a time of a pronuclei appearance, a time of a pronuclei disappearance, and/or one or more probabilities indicative of whether a particular number of pronuclei have appeared.

In some embodiments, the at least one trained machine learning model comprises a spatial transformer neural network and a multi-modal transformer neural network configured to process frame tokens output by the spatial transformer neural network, predicting the first degree of viability of the first embryo further comprises generating frame tokens representing the first sequence of image frames, the generating comprising processing the first sequence of image frames using the spatial transformer neural network to obtain the frame tokens, and processing the electronic health data and the first sequence of image frames using the at least one trained machine learning model to obtain the first degree of viability of the first embryo comprises processing the frame tokens and the electronic health data using the multi-modal transformer neural network to obtain the first degree of viability of the first embryo.

In some embodiments, generating the frame tokens representing the first sequence of image frames further comprises: processing the first sequence of image frames using the spatial transformer neural network to obtain spatial tokens for the first sequence of image frames; obtaining morphological feature tokens for the first sequence of image frames; and concatenating the spatial tokens and the morphological feature tokens to obtain the frame tokens.

In some embodiments, the at least one trained machine learning model further comprises a multilayer perceptron trained to predict a degree of viability of an embryo based on outputs generated by the multi-modal transformer neural network.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A and FIG. 1B are diagrams of illustrative techniques for selecting at least one embryo for transfer to a subject in furtherance of an in vitro fertilization (IVF) treatment, according to some embodiments of the technology described herein.

FIG. 2 is a flowchart of an illustrative process 200 for selecting at least one embryo for transfer to a subject in furtherance of an IVF treatment, according to some embodiments of the technology described herein.

FIG. 3 is a diagram of an example technique for predicting embryo viability using at least one machine learning model including a multi-modal machine learning model, according to a first embodiment of the technology described herein.

FIG. 4 shows an example image frame and example segmentation masks for the example image frame, according to some embodiments of the technology described herein.

FIG. 5 is a diagram of an example technique for predicting embryo viability using at least one machine learning model, according to a second embodiment of the technology described herein.

FIG. 6 is a schematic diagram of an illustrative computing device with which aspects described herein may be implemented.

### DETAILED DESCRIPTION

In-vitro fertilization (IVF) treatment entails transferring one or more fertilized embryos to the maternal uterus to initiate pregnancy. Although transferring multiple embryos might increase the likelihood of implantation, it also elevates the risk of multiple pregnancies, which are linked to heightened maternal and neonatal morbidity and mortality. Thus, to protect the health and safety of both the mother and the prospective pregnancy, it is important to limit the number of embryos transferred in furtherance of the IVF treatment. For example, it may be desirable to select a single embryo for transfer.

To limit the number of embryos for transfer without compromising the chance of achieving a successful pregnancy, it is important to be selective when choosing the embryo or embryos for transfer. In particular, it may be desirable to transfer the embryo(s) most likely to be viable. A viable embryo refers to an embryo that implants into the uterine wall.

The prevailing practice in embryo selection primarily relies on morphological analysis through microscopic imaging. Embryos undergo a series of developments post-fertilization, transitioning through stages from pronuclei alignment to blastocyst formation, with clinicians traditionally scoring embryos based on discrete, manually-observed morphokinetic features such as cell number, cell shape, cell symmetry, the presence of cell fragments, and blastocyst appearance. Some clinics adopt time-lapse microscopy incubators to capture movies of embryos continuously without disturbing their culture conditions. Despite this advancement, the analysis of these videos is performed manually by clinicians, which is labor-intensive, time-consuming, and subjective.

Computational techniques have been used to predict and analyze morphological and interpretable features of developing embryos using images or videos. For example, conventional computational techniques for predicting embryo viability rely on morphological features such as blastocyst size, blastocyst grade, cell boundaries, cell counting, and developmental stage prediction. When converted to interpretable features (e.g., timing of stage transitions, cell symmetry index, and zona thickness), these morphological features have been shown to be correlated to the live birth result of IVF treatments. However, these morphological and/or interpretable features may not capture more intricate and nuanced details of embryo development captured in videos, which in turn reduces the accuracy and reliability of conventional computational predictions that rely solely on these features.

Additionally, the conventional computational techniques for predicting embryo viability mainly focus on visual features and fail to account for various other important factors that also impact viability. In particular, the conventional computational techniques fail to account for the health and medical history of the patient, both of which have a significant impact on the prospective success of the pregnancy. For example, among other variables, age, IVF treatment information, and body mass index (BMI), are variables that impact embryo viability. By failing to account for such information, the conventional computational viability prediction techniques have reduces accuracy and reliability.

Accordingly, the inventors have developed techniques that address the above-described challenges associated with conventional computational techniques for predicting embryo viability. The embryo viability prediction techniques developed by the inventors utilize a multimodal machine learning approach that integrates both video data and electronic health data to inform accurate and reliable predictions of embryo viability.

Accordingly, in some embodiments, the embryo viability prediction techniques include: (a) obtaining video data for multiple embryos, (b) obtaining electronic health data for the subject, and (c) predicting, using the video data and the electronic health data, respective degrees of viability of at least some (e.g., all) of the multiple embryos. For example, the video data may include, for each embryo, a sequence of image frames depicting the embryo. The sequence of image frames and the electronic health data may be processed using at least one trained machine learning model to predict the viability of the particular embryo depicted in the image frames.

In some embodiments, the techniques developed by the inventors further include selecting at least one embryo for transfer to the subject using the predicted degrees of viability. For example, the embryo or embryos for which the highest degree(s) of viability were predicted may be selected for transfer. In some embodiments, the techniques further include transferring the selected embryo(s) and/or recommending (e.g., to a clinician) that they be transferred.

The techniques developed by the inventors constitute an improvement over conventional computational techniques for predicting embryo viability because they generate viability predictions that are more accurate and reliable, as a result of integrating data across multiple modalities in order to make the prediction. In particular, the techniques developed by the inventors make predictions of embryo viability by integrating: (i) video data that captures complex and nuanced morphological changes during embryo development, and (ii) electronic health data that captures information about the patient's health and the IVF treatment. Furthermore, utilizing at least one machine learning model to process the video data and electronic health data avoids subjective and manual analysis of video frames, further increases the accuracy and consistency of the resulting viability predictions, as well as the efficiency of the analysis.

The inventors have further recognized that, while there exist transformer models that can be used to process different data modalities, it is not straightforward to apply them to the task of embryo viability prediction, as they assume that samples in each modality have one-to-one correspondence. However, in the context of predicting embryo viability using video data and electronic health data, the samples in each modality are not one-to-one; video data is embryo-specific, while electronic health data is treatment specific. Thus, it is difficult to directly apply cross-modal correspondence or contrastive learning as in other multimodal learning approaches, and such approaches are not equipped to effectively handle such data. By contrast, the multimodal embryo viability prediction techniques described herein have been specifically designed to process video data and electronic health data for the purpose of predicting embryo viability. For example, some embodiments provide for a multi-modal transformer neural network that includes a video transformer (e.g., ViViT) modified to allow multi-modal inputs, thereby enabling the processing of the video data and electronic health data.

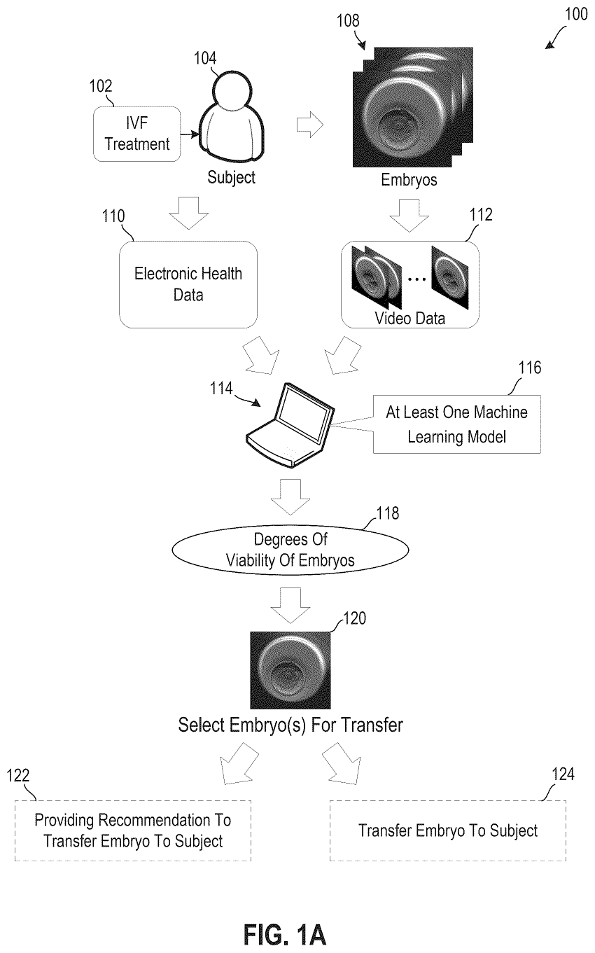

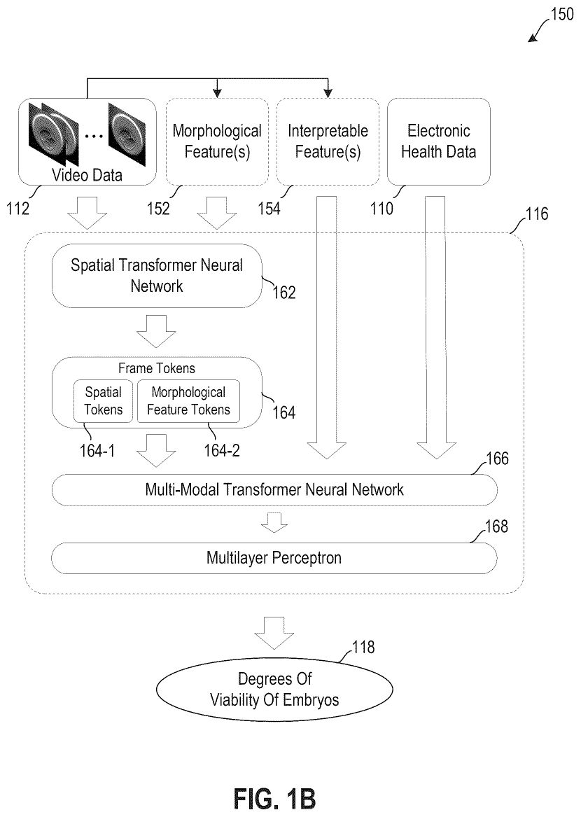

FIG. 1A is a diagram of an illustrative technique 100 for selecting at least one embryo for transfer to a subject 104 in furtherance of an in vitro fertilization (IVF) treatment 102, according to some embodiments of the technology described herein. As shown in FIG. 1A, technique 100 includes: (a) obtaining electronic health data 110 for the subject 104, (b) obtaining video data 112 for a plurality of embryos 108, (c) processing the electronic health data 110 and the video data 112 using computing device(s) 114 to obtain predictions 118 of respective degrees of viability of at least some of the embryos 108, and (d) using the predictions 118 to select embryo(s) 120 for transfer. In some embodiments, the technique 100 includes transferring the selected embryo(s) 120 to the subject 104 (e.g., act 124), and/or providing a recommendation to transfer the selected embryo(s) 120 to the subject 104 (e.g., act 122).

Subject 104 may undergo IVF treatment 102. During the IVF treatment 102, the subject 104 may be administered one or more medications (e.g., hormone medication(s)). One or more oocytes may be extracted from the subject, and the oocytes may be fertilized to obtain embryos 108. Embryos 108 may include between 2 and 15 embryos, or a number of embryos within any other suitable range, as aspects of the technology described herein are not limited in this respect.

In some embodiments, electronic health data 110 includes information about the IVF treatment 102 and/or information about subject 104. For example, the information about the IVF treatment 102 may include an indication of the fertilization type, an indication of a number of oocytes retrieved from the subject, an indication of medication(s) administered to the subject in furtherance of the IVF treatment, or any other suitable information about the IVF treatment, as aspects of the technology described herein are not limited in this respect. Information about the subject 104 may include an indication of one or more measurements of the subject such as measurements of hormone level(s) of the subject, a weight of the subject, a height of the subject, a body mass index (BMI) of the subject, an age of the subject, and/or any other suitable measurements, as aspects of the technology described herein are not limited in this respect. Additionally or alternatively, the information about the subject 104 may include information about the subject's medical history such as, for example, an indication of an age at which the subject first menstruated. Additional or alternative examples of electronic health data 110 are listed in Table 1.

In some embodiments, the video data 112 includes video data for each of at least some (e.g., all) of the embryos 108. The video data for a particular embryo may include a video depicting the embryo for at least part of the duration between fertilization and transfer of at least one of the embryos to the subject. In some embodiments, the video duration is at least 1 day, at least 2 days, at least 3 days, at least 4 days, at least 5 days, at least 6 days, at least 7 days, or at least any other suitable number of days, as aspects of the technology described herein are not limited in this respect. For example, the duration of the video may begin at the time of fertilization and capture at least the first 5 days of embryo development.

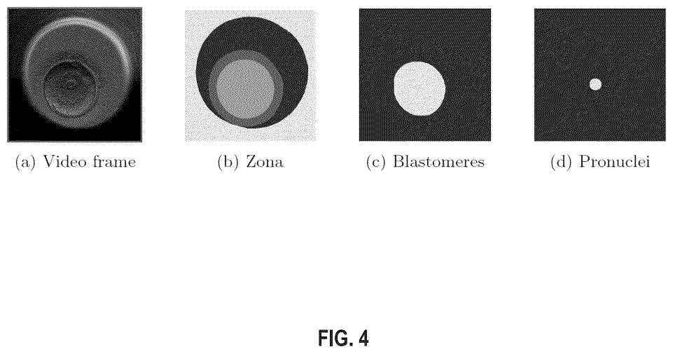

In some embodiments, the video data for a particular embryo may be a sequence of image frames depicting the embryo. The number of image frames depends on the duration of the video and the frequency at which image frames are captured. For example, the frequency may be between a frequency between 1 frame per hour and 120 frames per hour, or a frequency within any other suitable range, as aspects of the technology described herein are not limited in this respect. For example, the frequency may be 3 frames per hour (e.g., a frame captured every 10 minutes). An example image frame is shown in FIG. 4.

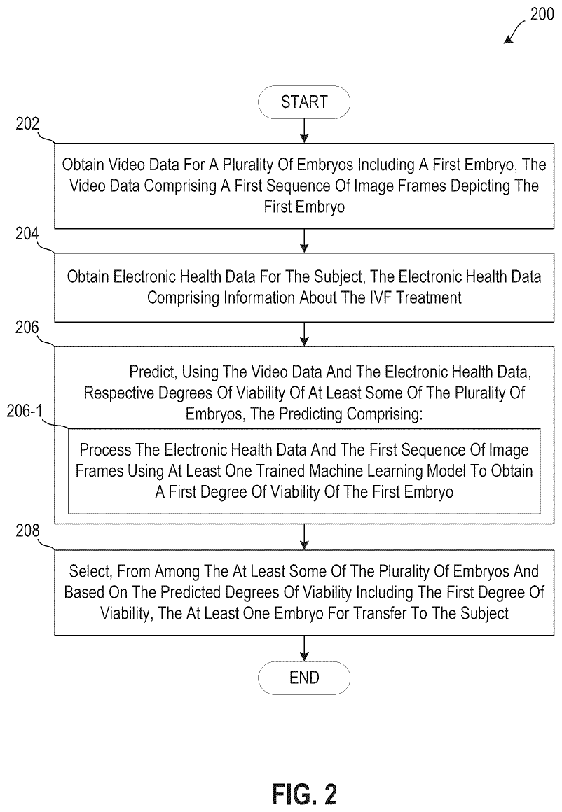

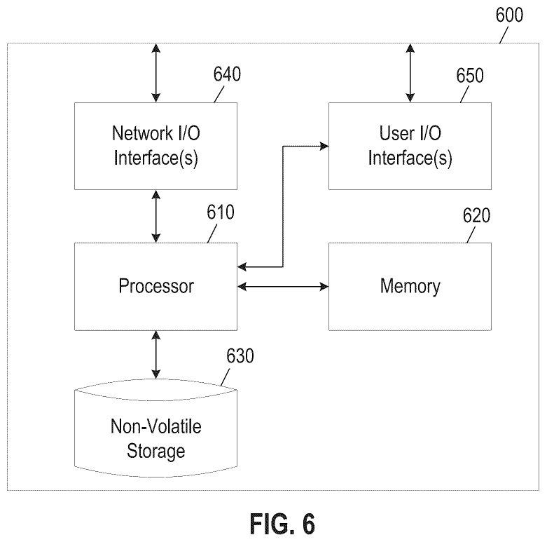

As shown in FIG. 1A, the electronic health data 110 and video data 112 are processed using computing device(s) 114. For example, computing device(s) 114 may include computing device 600 described herein including with respect to FIG. 6. In some embodiments, software executed on the computing device(s) 114 is configured to process the electronic health data 110 and video data 112 to predict respective degrees of viability of at least one, some, or all of the embryos 108. In some embodiments, this includes processing the electronic health data and a sequence of image frames depicting a particular embryo using at least one machine learning model 116 to obtain a degree of viability of the particular embryo. Example techniques for predicting embryo viability are described herein including at least with respect to illustrative technique 150 shown in FIG. 1B and process 200 shown in FIG. 2.

In some embodiments, the computing device(s) 114 output one or more predictions 118 of the respective degree(s) of viability of one or more of the embryos 108. Additionally or alternatively, the computing device(s) 114 may output a ranking of at least some of the embryos 108. For example, the embryos may be ranked according to the predicted degrees of viability. Additionally or alternatively, the computing device(s) 114 may output an indication of a recommendation for transferring at least one of the embryos 108 to subject 104.

As shown in FIG. 1A, illustrative technique 100 may additionally include selecting at least one embryo 120 for transfer to the subject 104. For example, the at least one embryo 120 may be selected from among embryos 108. In some embodiments, the selection is performed based on the predictions 118. Additionally or alternatively, the selection may be based on a recommendation generated by computing device(s) 114 for transferring at least one of the embryos to the subject 104. In some embodiments, embryo(s) predicted to have the highest degree of viability are selected for transfer. It should be appreciated that, though shown as separate from computing device(s) 114, the selection may be performed by computing device(s) 114.

In some embodiments, the technique 100 includes, at act 122, providing a recommendation to transfer the selected embryo(s) 120 to the subject 104. For example, a recommendation may be provided to a clinician, and the clinician may decide whether to transfer the selected embryo(s) 120 to the subject. The recommendation may be provided in any suitable format such as, for example, via a graphical user interface of computing device(s) 114.

Additionally or alternatively, illustrative technique 100 includes, at act 124, transferring the selected embryo(s) 120 to subject 104. For example, a clinician may transfer the embryo to the subject 104.

FIG. 1B is a diagram of an illustrative technique 150 of the processing performed by computing device(s) 114 for predicting respective degrees of viability of at least some of the embryos 108, according to some embodiments of the technology described herein. As shown in FIG. 1B, the video data 112 and electronic health data 110 is processed using at least one trained machine learning model 116 to obtain predictions 118 of respective degrees of viability of at least some of the embryos 108.

As shown in FIG. 1B, in addition to the video data 112 and electronic health data 110, one or more morphological features 152 and/or one or more interpretable features 154 may be processed using the at least one machine learning model 116 to obtain predictions 118.

In some embodiments, the morphological feature(s) 152 include features indicative of the morphology of the embryos 108 during development, prior to transfer to the subject. For example, the morphological feature(s) 152 may include features observable from the video data 112. Examples of morphological features of an embryo include: a segmentation of a zona pellucida, a grading of a degree of fragmentation, a classification of a developmental stage, an object instance segmentation of cells in a cleavage stage, and/or an object instance segmentation of pronuclei before a first cell division, and/or any other suitable type(s) of morphological features, as aspects of the technology described herein are not limited in this respect.

In some embodiments, the morphological feature(s) 152 are generated using the video data 112. For example, one or more (e.g., all) image frames included in a video of an embryo may be processed using at least one image processing technique to obtain morphological feature(s) for the embryo. In some embodiments, the image processing technique may be implemented using software configured to determine the morphological feature(s). Embryo-vision is an example of software configured to determine morphological feature(s) based on video data obtained for an embryo. Embryo-vision is described by Leahy, B. D., et al. (Automated measurements of key morphological features of human embryos for ivf. In: International Conference on Medical image computing and computer-assisted intervention. Springer (2020)), which is incorporated by reference herein in its entirety. However, it should be appreciated that any other suitable technique for determining morphological features may be used, as aspects of the technology described herein are not limited in this respect.

In some embodiments, the interpretable feature(s) 154 include features that are measurable by a human operator (e.g., a clinician). Like the morphological feature(s) 152, the interpretable feature(s) 154 may also include feature(s) indicative of the morphology of the embryos 108 during development. Examples of interpretable feature(s) 154 include a zona pellucida thickness, a standard deviation of the zona pellucida thickness, one or more diameters of an inner zona pellucida region, one or more diameters of an outer zona pellucida region, one or more transition times between embryo development stages, one or more fragmentation levels, a zygote size, a zygote shape, one or more cell symmetry indices, a time of a pronuclei appearance, a time of a pronuclei disappearance, one or more probabilities indicative of whether a particular number of pronuclei have appeared, and/or any other suitable type(s) of interpretable features, as aspects of the technology described herein are not limited in this respect. Additional or alternative examples of interpretable features are listed in Table 2.

In some embodiments, the interpretable feature(s) 154 are generated using video data 112 and/or morphological feature(s) 152. For example, the interpretable feature(s) 154 may be determined by processing the morphological feature(s) 152 using software configured to determine the interpretable feature(s) 154. BlastAssist is an example of software configured to determine interpretable feature(s). BlastAssist is described by Yang, H. Y., et al. (Blastassist: a deep learning pipeline to measure interpretable features of human embryos. Human Reproduction p. deac024 (2024)), which is incorporated by reference herein in its entirety. However, it should be appreciated that any other suitable technique for determining interpretable features may be used, as aspects of the technology described herein are not limited in this respect. For example, an operator may manually or semi-automatically determine one or more interpretable features using the video data 112 and/or morphological feature(s) 152.

As shown in FIG. 1B, technique 150 includes processing the video data 112 and/or morphological feature(s) 152 using spatial transformer neural network 162 to obtain frame tokens 164 representative of image frames included in the video data 112. The frame tokens 164, interpretable feature(s) 154, and/or electronic health data 110 are processed using multi-modal transformer neural network 166. The output of the multi-modal transformer neural network 166 is processed using the multilayer perceptron 168 to obtain the degrees of viability of the embryos (e.g., predictions 118). Because videos are typically significantly larger than the size of other modalities, directly apply spatio-temporal attention to a video may result in a large number of tokens, which would require an immense amount of memory and computation. By first applying the spatial transformer neural network 162, followed by the multi-modal transformer neural network 166, the techniques developed by the inventors help to conserve memory and reduce computation.

The spatial transformer neural network 162 may apply spatial attention to the video data 112 and/or morphological feature(s) 152 to obtain a plurality of frame tokens 164 representing the sequence image frames depicting a particular embryo. For example, the plurality of frame tokens 164 may include a respective frame token 164 for each of at least some (e.g., all) image frames included in sequence of image frames depicting the particular embryo. The spatial transformer neural network 162 may include any suitable neural network capable of performing spatial transformations, as aspects of the technology described herein are not limited in this respect. For example, the spatial transformer neural network 162 may include a sequence of transformer layers, each of which consists of multi-headed self-attention, layer normalization (LN), and MLP. For example, the spatial transformer neural network 162 may be one of the spatial encoders of the video vision transformer (ViViT) described by Arnab, A., et al. (A video vision transformer. In: IEEE International Conference on Computer Vision (2021)), which is incorporated by reference herein in its entirety.

In some embodiments, the input to the spatial transformer neural network 162 includes video data 112. For example, to predict the degree of viability of a particular embryo, the input video data may include one or more image frames of a sequence of image frames depicting the embryo. In some embodiments, prior to being provided as input to the spatial transformer neural network 162 each of the input image frames may be processed to obtain a respective initial image frame token (not shown). In some embodiments, an initial image frame token is generated for an image frame or a segmentation mask by (i) extracting image patches (e.g., non-overlapping image patches) from the image frame, and (ii) applying a linear projection to the image patches to obtain the initial image frame token.

In some embodiments, the spatial transformer neural network 162 processes the initial image frame tokens to obtain spatial tokens 164-1. For example, the set of initial image frame tokens and a learnable class token may be added to a learnable positional embedding and passed through the spatial transformer neural network 162 to obtain spatial tokens 164-1.

In some embodiments, the input to the spatial transformer neural network 162 additionally includes morphological feature(s) 152. For example, to predict the degree of viability of a particular embryo, the input morphological feature(s) may include for each of one or more image frames of a sequence of image frames depicting the embryo, one or more segmentation masks corresponding to the particular image frame. In some embodiments, prior to being provided as input to the spatial transformer neural network 162 each of the input segmentation masks may be processed to obtain a respective initial morphological feature token (not shown). In some embodiments, an initial morphological feature token is generated for a segmentation mask by (i) extracting patches (e.g., non-overlapping patches) from the segmentation mask, and (ii) applying a linear projection to the patches to obtain the initial morphological feature token.

In some embodiments, the spatial transformer neural network 162 processes the initial morphological feature tokens to obtain morphological feature tokens 164-2. For example, the set of initial morphological feature tokens and a learnable class token may be added to a learnable positional embedding and passed through the spatial transformer neural network 162 to obtain morphological feature tokens 164-2.

In some embodiments, the frame tokens 164 output by the spatial transformer neural network 162 include a respective frame token for each of the image frames for which an initial image frame token and/or an initial morphological feature token was generated. In some embodiments, when only video data 112 is provided as input to the spatial transformer neural network 162, the frame tokens 164 are the spatial tokens 164-1. When both video data 112 and morphological feature(s) 152 are provided as input, then the frame tokens 164 may include a concatenation of spatial tokens 164-1 and morphological feature tokens 164-2. For example, each of the frame tokens 164 may include a spatial token concatenated with a corresponding morphological feature token.

In some embodiments, the multi-modal transformer neural network 166 processes (i) frame tokens 164, (ii) electronic health data 110, and/or (iii) interpretable feature(s) 154. For example, the input to the multi-modal transformer neural network 166 may include the frame tokens 164 appended to the embedded electronic health data 110 and/or the embedded interpretable feature(s) 154. The electronic health data 110 and interpretable feature(s) 154 may be embedded by linear projection, for example. In some embodiments, the multi-modal input and a learnable class token are added to a learnable temporal embedding and passed through the multi-modal transformer neural network.

The multi-modal transformer neural network 166 may apply temporal attention to the multi-modal input. The multi-modal transformer neural network 166 may include a video transformer (e.g., ViViT) modified to allow multi-modal inputs. For example, the multi-modal transformer neural network 166 may have the architecture described in Table 5.

In some embodiments, the output of the multi-modal transformer neural network 166 is processed by MLP 168 to obtain a predicted degree of viability of the particular embryo for which the input data was provided. In some embodiments, MLP 168 includes two fully connected layers with ReLU activation in between.

In some embodiments, a degree of viability output by MLP 168 is a likelihood that an embryo will be viable if transferred to a subject. For example, the output of the MLP 168 may indicate a probability that the embryo will be viable if transferred to the subject. Additionally or alternatively, the output may indicate a classification for the embryo. For example, the classification may be a binary classification indicating whether or not the embryo is likely to be viable if transferred to a subject.

FIG. 2 is a flowchart of an illustrative process 200 for selecting at least one embryo for transfer to a subject in furtherance of an IVF treatment, according to some embodiments of the technology described herein. One or more of acts of process 200 may be performed automatically by any suitable computing device(s). For example, act(s) may be performed by computing device(s) 114 shown in FIG. 1A, computing device 600 shown in FIG. 6, a laptop computer, a desktop computer, a mobile device, one or more servers, in a cloud computing environment, and/or in any other suitable way, as aspects of the technology described herein are not limited in this respect.

At act 202, video data is obtained for a plurality of embryos including a first embryo. In some embodiments, the video data includes a first sequence of image frames depicting the first embryo. Examples of video data and techniques for obtaining same are described herein including at least with respect to techniques 100 and 150 shown in FIG. 1A and FIGS. 1B and 1n the section entitled “EXAMPLES”. For example, the video data may include video data 112 shown in FIG. 1A and FIG. 1B.

At act 204, electronic health data is obtained for the subject. In some embodiments, the electronic health data includes information about the IVF treatment. Examples of electronic health data and techniques for obtaining same are described herein including at least with respect to techniques 100 and 150 shown in FIG. 1A and FIG. 1B and in the section entitled “EXAMPLES”. For example, the electronic health data may include electronic health data 110 shown in FIG. 1A and FIG. 1B.

At act 206, respective degrees of embryo viability are predicted for at least some of the plurality of embryos using the video data and the electronic health data. In some embodiments, predicting the respective degrees of viability includes, at act 206-1, processing the electronic health data and the first sequence of image frames using at least one machine learning model to obtain a first degree of viability of the first embryo. Examples of techniques for predicting embryo viability are describe herein including at least with respect to techniques 100 and 150 shown in FIG. 1A and FIG. 1B and in the section entitled “EXAMPLES”. For example, degrees of viability may be predicted according to illustrative technique 150 shown in FIG. 1B, by processing the obtained video data and electronic health data using at least one trained machine learning model.

At act 208, at least one embryo is selected for transfer based on the predicted degrees of viability including the first degree of viability of the first embryo. Example techniques for selecting at least one embryo for transfer are described herein including at least with respect to illustrative technique 100 shown in FIG. 1A and in the section entitled “EXAMPLES”.

### EXAMPLES

This example relates to a multimodal model that leverages both time-lapse video data and Electronic Health Records (EHRs) to predict embryo viability. This example includes the following sections: “Dataset,” “Method,” and “Experiments.”

### Dataset

Data is collected from 3,695 IVF treatment cycles with 24,027 embryos imaged every 20 minutes up to the first five days of development where the image size is 500×500. This corresponds to approximately 6 million images of embryos. Additionally, electronic health record (EHR) data, including patient information, treatment information, and live birth records as a treatment outcome, are collected. Among the collected data samples, a multimodal dataset is curated with embryos that have both video and EHR modalities with treatment out-comes. The multimodal dataset comprises 1700 treatment cycles with 3318 embryos. Out of 1700 treatments, 260 treatments are successful with equal or more than one live birth. A treatment cycle fertilizes multiple embryos, and only healthy embryos are selected for transfer. Some cycles freeze all embryos for future use rather than immediate transfer. Therefore, the number of embryos that have the treatment outcome is limited compared to the scale of the raw data collected.

### Method

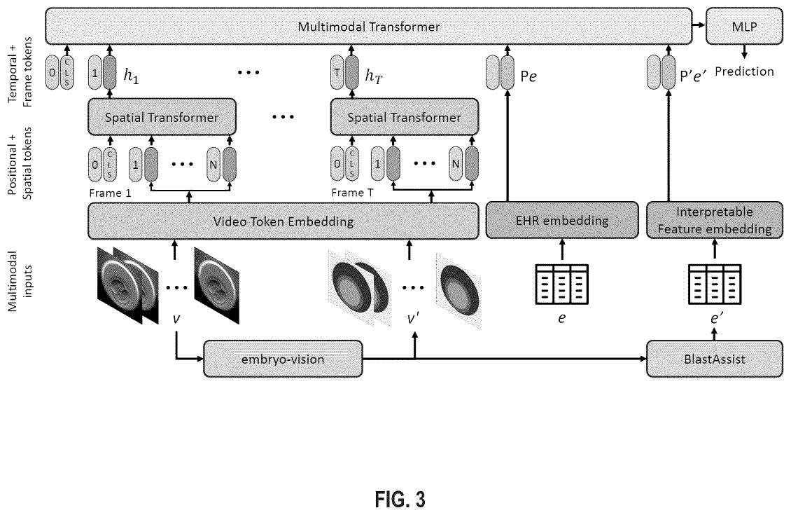

Two different directions to integrate multimodal data for embryo viability prediction are explored. One is a transformer-based multimodal model where EHRs and videos are processed end-to-end, as shown in FIG. 3. The multimodal transformer is based on a video transformer architecture with modifications to allow multimodal inputs. Video data is first tokenized into patches per frame. Then, the spatial transformer encodes per frame embeddings. The Multimodal transformer inputs both frame embeddings and an EHR embedding to output a multimodal feature. Lastly, the MLP head predicts embryo viability based on the multimodal feature. If additional inputs in the form of video or tabular are available, such as outputs from Embryo-vision or BlastAssist, they are processed in a similar manner as the video input and the EHR input respectively.

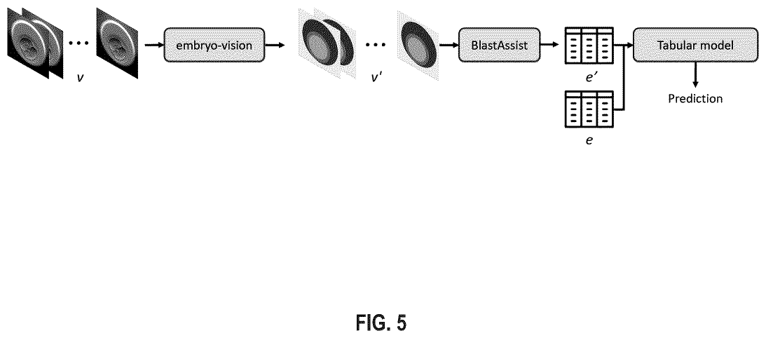

Another approach is to take a two-stage approach where the video data is first processed to extract morphological features in tabular format using off-the-shelf methods, and then input to the tabular models with EHRs as shown in FIG. 5. The two-stage approach is multimodal by nature as video data is converted and included in a tabular format. First, morphological features v′ are extracted from videos using Embryo-vision. Then, the extracted features v′ are converted to interpretable features e′ in tabular format using BlastAssist. Lastly, the tabular model inputs EHRs e and interpretable features e′ to predict embryo viability.

### Input Modalities

Let τₙ={vₙ, eₙ} be a multimodal sample in n-th treatment cycle in the multimodal dataset, where

$${v}_{n}^{m} ∈ {ℝ}^{T \times H \times W \times C}$$

denotes a time-lapse video of m-th embryo fertilized in n-th treatment cycle and eₙ∈ [symbol] ^{C} denotes an EHR containing information of the patient and treatment applied. Time-lapse videos are embryo-specific, but EHR data corresponds to the treatment cycle; thus, they are not embryo-specific. Embryo viability is formulated as

$$y = \frac{n\_births}{n\_transferred} ,$$

where viability is defined as the number of births over the number of embryos transferred. The number of embryos transferred at a treatment cycle varies depending on various factors, such as the number of embryos fertilized, embryo quality examined by embryologists, or the patient's medical history. Examples of EHR data are listed in Table 1.

TABLE 1
EHR data columns. Columns marked as ‘Index’ are used to curate a dataset
and splits. Columns marked as ‘Input’ are used as a multimodal model input.
Columns marked as ‘Output’ are used to generate ground truth for training and evaluation.

| Usage | Column Name | Data Type | Description |
|---|---|---|---|
| Index | Patient Number | int | Unique patient ID |
| Index | Treatment ID | string | Index of a treatment |
| Index | Well ID | int | Index of an embryo within a treatment cycle |
| Index | Transferred | int | Whether an embryo is transferred or not |
| Input | Patient age | float | Age of a patient |
| Input | Patient BMI | float | BMI of a patient |
| Input | Age Of First Menstrual | float | Age Of First Menstrual |
| Input | Total Retrieved Oocytes | int | Total number of oocytes retrieved for treatment |
| Input | Fertilization Type | string | Type of the treatment. Converted to the class label |
| Input | e2-1 | int | E2 hormone level at day 1 |
| Input | e2-2 | int | E2 hormone level at day 2 |
| Input | e2-3 | int | E2 hormone level at day 3 |
| Output | Total number embryos | int | Total number of embryos fertilized |
| Output | Children N | int | Number of children born |

Other than video data, morphological embryo features are also utilized. The morphological embryo features are extracted from videos by off-the-shelf methods, e.g., Embryo-vision and BlastAssist. Embryo-vision outputs a set of features

$${v}_{n , t}^{′ m}$$

a video frame

$${v}_{n , t}^{m}$$

which are zona semantic segmentation s_{z}, blastomere instance segmentation s_{b}, pronuclei instance segmentation sₚ, fragmentation regression r, and stage classification c. FIG. 4 provides a visualization of the Embryo-vision outputs for semantic segmentation (zona), and instance segmentation (blastomeres and pronuclei). Fragmentation prediction is a float value, and stage prediction is a 13-dimensional vector where each dimension represents the probability of each stage. BlastAssist further converts the morphological features into a set of interpretable features e′ such as zona well thickness, stage transition timing, and cell symmetry index. Examples of BlastAssist features are listed in Table 2.

TABLE 2
BlastAssist columns. Columns marked as ‘Index’ are used to curate a dataset
and splits. Columns marked as ‘Input’ are used as a multimodal model input.
Columns marked as ‘Output’ are used to generate ground truth for training and evaluation.

| Usage | Column Name | Data Type | Description |
|---|---|---|---|
| Index | Patient Number | int | Unique patient ID |
| Index | Treatment ID | string | Index of a treatment |
| Index | Well ID | int | Index of an embryo within a treatment cycle |
| Index | Transferred | int | Whether an embryo is transferred or not |
| Input | zona width mean | float | Average zona well thickness |
| Input | zona width std | float | Standard deviation of zona well thickness |
| Input | zona inner diameter max | float | Max diameter of an inner zona region |
| Input | zona inner diameter min | float | Min diameter of an inner zona region |
| Input | zona outer diameter max | float | Max diameter of an outer zona region |
| Input | zona outer diameter min | float | Min diameter of an outer zona region |
| Input | frag day 2 median | float | Median fragmentation level on day 2 |
| Input | frag day 3 median | float | Median fragmentation level on day 3 |
| Input | 2-cell time | float | Transition time to 2-cell stage |
| Input | 3-cell time | float | Transition time to 3-cell stage |
| Input | 4-cell time | float | Transition time to 4-cell stage |
| Input | 5-cell time | float | Transition time to 5-cell stage |
| Input | 6-cell time | float | Transition time to 6-cell stage |
| Input | 7-cell time | float | Transition time to 7-cell stage |
| Input | 8-cell time | float | Transition time to 8-cell stage |
| Input | 9+-cell time | float | Transition time to 9+-cell stage |
| Input | morula time | float | Transition time to morula stage |
| Input | blastocyst time | float | Transition time to blastocyst stage |
| Input | zygote area | float | Size of zygote |
| Input | zygote shape | float | Shape parameter of zygote |
| Input | 2-cell symmetry | float | Cell symmetry index at 2-cell stage |
| Input | 4-cell symmetry | float | Cell symmetry index at 4-cell stage |
| Input | pn appear time | float | Time when pronuclei appears |
| Input | pn fade time | float | Time when pronuclei disappears |
| Input | prob 0 pn | float | Probability of 0 pronucleus appeared |
| Input | prob 1 pn | float | Probability of 1 pronucleus appeared |
| Input | prob 2 pn | float | Probability of 2 pronucleus appeared |
| Input | prob 3+ pn | float | Probability of 3 or more pronuclei appeared |
| Output | Total number embryos | int | Total number of embryos fertilized |
| Output | Children N | int | Number of children born |

### Video Transformer

In this example, a transformer is designed in a factorized encoder structure where spatial attention is applied first, followed by temporal attention.

For spatial attention, a frame (e.g., each frame)

$${v}_{n , t}^{m} ∈ {ℝ}^{H \times W \times C}$$

is first tokenized to a set of tokens by extracting non-overlapping image patches xᵢ∈ [symbol] ^{h×w×c}. A linear projection E is then applied. Then, a set of embedded frame tokens and a learnable class token are added to a learnable positional embedding p and passed through a transformer comprising a sequence of L transformer layers to output a frame-level representation.

$$z = [ {z}_{cls} , {Ex}_{1} , ... , {Ex}_{N} ] + p \qquad ( Equation 1 )$$

A transformer layer l (e.g., each transformer layer l) comprises Multi-Headed Self-Attention, layer normalization (LN), and MLP blocks as follows:

$${y}^{l} = MSA ( LN ( {z}^{l} ) ) + {z}^{l} \qquad ( Equation 2 )$$

$${z}^{l + 1} = MLP ( LN ( {y}^{l} ) ) + {y}^{l} \qquad ( Equation 3 )$$

The output token

$${z}_{cls}^{L}$$

embeds frame-level representation. Temporal attention is performed similarly to spatial embedding by applying L′ transformer layers on a set of frame tokens h,

$$h = [ {h}_{cls} , {z}_{cls , 1}^{L} , ... , {z}_{cls , T}^{L} ] + t \qquad ( Equation 4 )$$

where h_{cls} is a learnable class token in temporal attention, and t is a learnable temporal embedding.

### Multimodal Transformer

A video transformer is modified to allow multi-modal inputs. EHR data e is embedded by linear projection and then append to the frame tokens. Additional features in a tabular format, e.g., interpretable features e′, are processed in the same way as EHR data. With EHR data tokens, the temporal attention input in Eq. (4) becomes multimodal attention input as follows,

$$h = [ {h}_{cls} , {h}_{1} , ... , {h}_{t} , {P}_{e} , {{P}^{′}}_{{e}^{′}} ] + t \qquad ( Equation 5 )$$

where hₜ is a frame token at frame t, P and P′ are linear projections for e and e′ respectively. When only video is input to the model, a frame token hₜ becomes

$${z}_{cls , t}^{L}$$

as in Eq. (4). Additionally, more per-frame modality inputs can be incorporated from Embryo-vision to enrich the representation of a frame token hₜ. The Embryo-vision outputs a set of morphological features v′={s_{z}, s_{b}, sₚ, r, c} where the first three features are segmentation masks and the latter two are vectors. The mask format features are passed to the spatial attention and processed similarly to the video input. For simplicity, let's denote spatial transformer operation fₛ: [symbol] ^{H×W×C}→ [symbol] ^{d}. When a video is input, fₛ(vₜ) equals

$${z}_{cls , t}^{L}$$

as in Eq. (4). When multiple video modalities are available, the frame token hₜ is formulated as a concatenation of tokens from different modalities as follows,

$${h}_{t} = [ {f}_{s} ( {v}_{t} ) , {f}_{s} ( {s}_{z} , t ) , {f}_{s} ( {s}_{b} , t ) , {f}_{s} ( {s}_{p} , t ) , {E}^{′} [ {r}_{t} , {c}_{t} ] ] \qquad ( Equation 6 )$$

where E′ is a linear projection applied to the concatenation of rₜ and cₜ.

TABLE 3
Number of successful and failed treatments and
embryos in each split in the form of “number
of embryos”/“number of treatments.”

| Split | Total | Success | Fail |
|---|---|---|---|
| Train | 2617/1360 | 362/208 | 2255/1152 |
| Validate | 327/170 | 54/26 | 273/144 |
| Test | 342/170 | 54/26 | 288/144 |

### Experiments

### Data Format

TABLE 4
Data formats.

| Data Type | Dimensions |
|---|---|
| EHR | 8 dimensions |
| HER-CV (BlastAssist) | 39 dimensions |
| Video | t × 1 × 500 × 500 |
| | (frame length t varies |
| | between 100-500) |
| Embryo-vision Video | t × 3 × 500 × 500 |
| | (frame length t varies |
| | between 100-500) |

### Data Preprocessing

The video data for a particular embryo has the dimensions: t×1×500×500, where the frame length t varies between 100-500. First, t is clipped to 360 frames (e.g., t×1×500×500→360×1×500×500), since this corresponds to the first 5 days of observation, where a frame (e.g., each frame) is captured at 20-minute intervals. If t is less than 360, t is padded with zeros. To enable memory-efficient training, every 4 frames are subsampled, resulting in 90 frames per video (e.g., 360×1×500×500→90×1×500×500). Third, the frame size is resized to the model input size (e.g., 90×1×500×500→90×1× 224× 224).

The morphological feature (e.g., Embryo-vision) video data (e.g., frame masks) has the dimensions: t×3×500×500, where the frame length t varies between 100-500. The morphological feature video data is pre-processed in the same manner as the video data. For example, the dimensions of the Embryo-vision video data may be reduced to: 90×3×224×224.

The morphological feature (e.g., Embryo-vision) non-video feature data has the following dimensions: t×3×(13+1), where t refers to the number of frames, there are 3 focal settings. The stage prediction feature is a 13-dimensional vector, while the fragmentation prediction feature is a float value. First, t is clipped to 360 frames (e.g., t×3×(13+1)→360×3× (13+1)). If t is less than 360, t is padded with zeros. Second, a frame is sampled every four frames to reduce computational resources (e.g., 360×3×(13+1))→90×3×(13+1)). Finally, the features are averaged across the focal settings (e.g., 90×3× (13+1)→90×1×(13+1)).

The data is augmented by applying random rotations and flips.

### Implementation Details

For spatial attention, the pre-trained DeiT-Ti was used as a spatial transformer without fine-tuning. For temporal or multimodal attention, 4 transformer layers are used. The architecture of the multimodal model is described in Table 5. MLP head consists of two fully connected layers with ReLU activation in between.

TABLE 5
Multimodal model architecture. The variable m in MLP and Multimodal transformer
represents a number of available tokens to concatenate. If all modalities are used,
then m is set to 5. (1 token from a video and 4 tokens from embryo-vision.)

| Component | Layer | Dimension | Kernel | Stride | Padding |
|---|---|---|---|---|---|
| EHR | LayerNorm | 8 | — | — | — |
| Embedding | Linear | 8 | 8 × 192 | — | — |
| | LayerNorm | 192 | — | — | — |
| Interpretable | LayerNorm | 39 | — | — | — |
| Feature | Linear | 39 | 39 × 192 | — | — |
| Embedding | LayerNorm | 192 | — | — | — |
| Video Token Embedding & Spatial Transformer | | | | deit_tiny_patch16_224 | |
| MLP | Linear | 192 × m | (192 × m) × 1 | | |
| Component | Input_dim | Depth | Num_heads | Head_dim | FF_dim |
| Multimodal | 192 × m | 4 | 8 | 64 | 256 |

### Experiment Setup

Train, validation, and test splits are randomly split to an 8:1:1 ratio while preserving the success rate within each split. For training and evaluation, the batch size is set to 4, the learning rate is set to 1e-4, and the model is trained until the validation loss converges. MLP head consists of two fully connected layers with ReLU activation in between. Huber loss is used to train the multimodal transformer. The experiments are performed using one A100 GPU. The training and evaluation settings are listed in Table 6. The training algorithm is described in Table 7.

For evaluation, two performance metrics were used: the area under the receiver operating characteristic curve (ROCAUC) and F1-Score. Two different scenarios were evaluated: embryo viability prediction and treatment success prediction. Each treatment has equal to, or more than one embryo transferred. In the embryo viability pre-diction scenario, the ground truth label is set to ‘1’ for all embryos transferred (instead of

$$\frac{n\_births}{n\_transferred} )$$

if the treatment is successful, then AUCROC and F1-Score are computed. In treatment success prediction, the viability predictions of embryos transferred together are summed, and then AUCROC and F1-Score are calculated. For F1-Score measurement, 0.15 is used as a threshold for embryo viability prediction and 0.5 is used as a threshold for treatment success prediction. F1-Score quantifies the precision of predictions at a fixed threshold, whereas AUCROC measures capability in assessing the relative quality of the samples.

TABLE 6
Training and evaluation settings.
Training and Evaluation Settings

| | |
|---|---|
| Batch Size | 4 |
| Max Epochs | 10 |
| Learning Rate | 1e−4 |
| Weight Decay | 0 |
| Optimizer | Adam |
| Loss Function | Huber loss |
| δ | 0.2 |

TABLE 7
Algorithm for training the multimodal model.
Algorithm Multimodal model training

| Input: | fₛ: spatial transformer, f_{M}: multimodal transformer, fₑ: EHR encoder, fᵢ: | |
|---|---|---|
| | interpretable feature encoder, f_{c}: classifier, v: video, v′: embryo-vision, e: EHR, | |
| | e′: interpretable, y: label, D: training set | |
| Output: | Updated f_{M}, fₑ, fᵢ, f_{c} | |
| for | v, v′, e, e′, y in D do | [symbol] Sample a mini-batch |
| | v, v′ ← aug(v), aug(v′) | |
| | with no_grad( ): | [symbol] Freeze fₛ |
| | V, V′ ← fₛ (v), fₛ(v′) | |
| | V ← V\|\|V′ | [symbol] Concatenation |
| | E, E′ ← fₑ (e), fᵢ(e′) | |
| | h ← f_{M} (V, E, E′) | |
| | \tilde{y} ← f_{c} (h) | [symbol] Prediction |
| | L← L_{huber} (\tilde{y}, y), | [symbol] huber loss |
| | L.backward( ) | [symbol] Back-propagate |
| | update(f_{M}, fₑ, fᵢ, f_{c}) | [symbol] Adam update |
| end for | | |

### Two-Stage Approach

The multimodal transformer is compared with two-stage approaches using two transformer-based methods: TabTransformer and Tab-Net. The tabular modules were trained according to the implementation described by Cui, W. (Mother or nothing: the agony of infertility. World Health Organization. Bulletin of the World Health Organization 88(12), 881 (2010)), which is incorporated by reference herein in its entirety. The hyperparameters were selected after performing a hyperparameter search using cross-validation.

Experiments with Multimodal Transformer

The multimodal transformer is evaluated on embryo viability prediction task using different combinations of modalities in Table 8. The first 4 rows in the table show the results with video modality. The model trained with only video modality performs worse than the other modality combinations. When both video and EHR modalities are used, AUCROC marginally improves. On the other hand, the model performance improves significantly when semantic features are added. This shows that directly predicting embryo viability is challenging and semantic information is important for the prediction. However, adding tabular format modalities to video modalities did not improve the prediction. This may be due to the increased complexity of multimodal data to learn given limited training samples. The performance drop with interpretable features is noticeable with video modality, but the performance drop is not observed in other combinations of modalities.

The multimodal model is evaluated without a video input v in the last 2 rows in Table 8. The results without a video modality perform better than those with a video modality. This may be due to the limited number of training videos to learn good representation. A pre-trained vision transformer DeiT-Ti is deployed to overcome the limited training set size, but multimodal transformer layers are trained from scratch; therefore, the multimodal attention is performed in a sub-optimal way. On the contrary, a model trained with Embryo-vision outputs v′ performs significantly better than those with v. Unlike raw video, Embryo-vision outputs are in the form of segmentation masks, which are semantically meaningful and have a simple visual structure. Therefore, it is easier for the model to understand and optimize the weights to extract relevant features for the task.

TABLE 8
Performance comparison on embryo viability prediction with different
modalities using a multimodal transformer. v is a video modality,
v′ is an output from Embryo-vision, e is EHR data, and e′
is an output from BlastAssist. The best performance is marked in bold.

| | |
|---|---|
| Embryo | Treatment |

| Modality | AUCROC | F-1 | AUCROC | F-1 |
|---|---|---|---|---|
| v | 0.578 | 0.284 | 0.579 | 0.315 |
| v + e | 0.580 | 0.297 | 0.581 | 0.286 |
| v + v′ | 0.676 | 0.316 | 0.675 | 0.336 |
| v + v′ + e + e′ | 0.647 | 0.296 | 0.643 | 0.310 |
| v | 0.666 | 0.317 | 0.697 | 0.313 |
| v′ + e + e′ | 0.688 | 0.338 | 0.683 | 0.312 |

Experiments with Two-Stage Approach

The two-stage approach is compared with different types of tabular models. The results are shown in Table 9. Unlike the end-to-end multimodal learning method, higher performance variation is observed in two-stage methods. This may be due to the early convergence of two-stage models, which results in different solutions. Here, confidence intervals are reported from 10 trials of the two-stage approaches. Among different modalities, using both EHR and interpretable features performs best for the two-stage approaches. Although visual data is not directly input to the model, interpretable features encode visual information; therefore, the tabular models show competitive performance when using both EHRs and interpretable features.

One noticeable difference to the multimodal transformer is the low F-1 score on treatment success prediction. Although tabular models are trained with regression objectives, they fail to calibrate the prediction confidence, resulting in a low F-1 score. In practice, finding the best threshold can be challenging. Therefore, without an appropriate threshold estimation method, a model with good confidence calibration is favored. If an optimal threshold can be found, a higher F-1 score will be achieved for both multimodal transformers and two-stage tabular models.

TABLE 9
Performance comparison on embryo viability prediction with different
modalities using a two-stage approach. e is EHR data, and e′ is
an output from BlastAssist. Confidence intervals are reported with 10 runs.

| | | Embryo | | Treatment | |
|---|---|---|---|---|---|
| Modality | Method | AUCROC | F-1 | AUCROC | F-1 |
| e | TabTransformer | 0.586 ± 0.045 | 0.110 ± 0.068 | 0.604 ± 0.054 | 0.167 ± 0.111 |
| | TabNet | 0.591 ± 0.016 | 0.240 ± 0.020 | 0.631 ± 0.017 | 0.113 ± 0.033 |
| e + e′ | TabTransformer | 0.634 ± 0.025 | 0.298 ± 0.045 | 0.681 ± 0.023 | 0.100 ± 0.031 |
| | TabNet | 0.629 ± 0.025 | 0.244 ± 0.042 | 0.672 ± 0.026 | 0.188 ± 0.058 |
| e′ | TabTransformer | 0.593 ± 0.021 | 0.235 ± 0.040 | 0.624 ± 0.022 | 0.134 ± 0.030 |
| | TabNet | 0.623 ± 0.012 | 0.232 ± 0.042 | 0.630 ± 0.023 | 0.146 ± 0.045 |

### Computer Implementation

An illustrative implementation of a computer system 600 that may be used in connection with any of the embodiments of the technology described herein (e.g., such as the process 200 shown in FIG. 2) is shown in FIG. 6. The computer system 600 includes one or more processors 610 and one or more articles of manufacture that comprise non-transitory computer-readable storage media (e.g., memory 620 and one or more non-volatile storage media 630). The processor 610 may control writing data to and reading data from the memory 620 and the non-volatile storage media 630 in any suitable manner, as the aspects of the technology described herein are not limited to any particular techniques for writing or reading data. To perform any of the functionality described herein, the processor 610 may execute one or more processor-executable instructions stored in one or more non-transitory computer-readable storage media (e.g., the memory 620), which may serve as non-transitory computer-readable storage media storing processor-executable instructions for execution by the processor 610.

Computing system 600 may include a network input/output (I/O) interface 640 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing system 600 may also include one or more user I/O interfaces 650, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as an example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as an example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for selecting at least one embryo for transfer to a subject in furtherance of an in vitro fertilization (IVF) treatment, the method comprising:
using at least one processor to perform:
obtaining video data for a plurality of embryos including a first embryo, the video data comprising a first sequence of image frames depicting the first embryo;
obtaining electronic health data for the subject, the electronic health data comprising information about the IVF treatment;
predicting, using the video data and the electronic health data, respective degrees of viability of at least some of the plurality of embryos, the predicting comprising:
processing the electronic health data and the first sequence of image frames using at least one trained machine learning model to obtain a first degree of viability of the first embryo; and
selecting, from among the at least some of the plurality of embryos and based on the predicted degrees of viability including the first degree of viability, the at least one embryo for transfer to the subject.

2. The method of claim 1, further comprising:
after selecting the at least one embryo for transfer, transferring the at least one embryo to the subject.

3. The method of claim 1, further comprising:
after selecting the at least one embryo for transfer, generating a recommendation to transfer the at least one embryo to the subject; and
providing an indication of the recommendation to a user.

4. The method of claim 1, wherein the information about the IVF treatment comprises an indication of a fertilization type and/or an indication of a number of oocytes retrieved from the subject.

5. The method of claim 1, wherein the electronic health data further comprises an indication of one or more measurements of the subject, the one or more measurements comprising measurements of one or more hormone levels of the subject, a weight of the subject, a height of the subject, a body mass index (BMI) of the subject, and/or an age of the subject.

6. The method of claim 1, wherein the electronic health data further comprises information about a medical history of the subject.

7. The method of claim 6, wherein the information about the subject's medical history comprises an indication of an age at which the subject first menstruated.

8. The method of claim 1, further comprising:
generating, using the video data, morphological features for the at least some of the plurality of embryos,
wherein predicting the respective degrees of viability of the at least some of the plurality of embryos comprises predicting the respective degrees of viability based on the electronic health data, the video data, and the morphological features.

9. The method of claim 8,
wherein the morphological features comprise one or more morphological features for the first embryo, and
wherein predicting the first degree of viability of the first embryo comprises:
processing the electronic health data, the first sequence of image frames, and the one or more morphological features using the at least one trained machine learning model to obtain the first degree of viability of the first embryo.

10. The method of claim 8, wherein the morphological features comprise, for each of the at least some of the plurality of embryos, a segmentation of a zona pellucida, a grading of a degree of fragmentation, a classification of a developmental stage, an object instance segmentation of cells in a cleavage stage, and/or an object instance segmentation of pronuclei before a first cell division.

11. The method of claim 1, further comprising:
obtaining interpretable features for the at least some of the plurality of embryos,
wherein predicting the respective degrees of viability of the at least some of the plurality of embryos comprises predicting the respective degrees of viability based on the electronic health data, the video data, and the interpretable features.

12. The method of claim 11,
wherein the interpretable features comprise one or more interpretable features for the first embryo, and
wherein predicting the first degree of viability of the first embryo comprises:
processing the electronic health data, the first sequence of image frames, and the one or more interpretable features using the at least one trained machine learning model to obtain the first degree of viability of the first embryo.

13. The method of claim 11, wherein the interpretable features comprise, for each of the at least some of the plurality of embryos, a zona pellucida thickness, a standard deviation of the zona pellucida thickness, one or more diameters of an inner zona pellucida region, one or more diameters of an outer zona pellucida region, one or more transition times between embryo development stages, one or more fragmentation levels, a zygote size, a zygote shape, one or more cell symmetry indices, a time of a pronuclei appearance, a time of a pronuclei disappearance, and/or one or more probabilities indicative of whether a particular number of pronuclei have appeared.

14. The method of claim 1,
wherein the at least one trained machine learning model comprises a spatial transformer neural network and a multi-modal transformer neural network configured to process frame tokens output by the spatial transformer neural network,
wherein predicting the first degree of viability of the first embryo further comprises generating frame tokens representing the first sequence of image frames, the generating comprising processing the first sequence of image frames using the spatial transformer neural network to obtain the frame tokens, and
wherein processing the electronic health data and the first sequence of image frames using the at least one trained machine learning model to obtain the first degree of viability of the first embryo comprises processing the frame tokens and the electronic health data using the multi-modal transformer neural network to obtain the first degree of viability of the first embryo.

15. The method of claim 14,
wherein generating the frame tokens representing the first sequence of image frames further comprises:
processing the first sequence of image frames using the spatial transformer neural network to obtain spatial tokens for the first sequence of image frames;
obtaining morphological feature tokens for the first sequence of image frames; and
concatenating the spatial tokens and the morphological feature tokens to obtain the frame tokens.

16. The method of claim 14, wherein the at least one trained machine learning model further comprises a multilayer perceptron trained to predict a degree of viability of an embryo based on outputs generated by the multi-modal transformer neural network.

17. A system, comprising:
at least one processor; and
at least one non-transitory computer-readable storage medium storing processor-executable instructions that, when executed by the at least one processor, cause the at least one processor to perform a method for selecting at least one embryo for transfer to a subject in furtherance of an in vitro fertilization (IVF) treatment, the method comprising:
obtaining video data for a plurality of embryos including a first embryo, the video data comprising a first sequence of image frames depicting the first embryo;
obtaining electronic health data for the subject, the electronic health data comprising information about the IVF treatment;
predicting, using the video data and the electronic health data, respective degrees of viability of at least some of the plurality of embryos, the predicting comprising:
processing the electronic health data and the first sequence of image frames using at least one trained machine learning model to obtain a first degree of viability of the first embryo; and
selecting, from among the at least some of the plurality of embryos and based on the predicted degrees of viability including the first degree of viability, the at least one embryo for transfer to the subject.

18. At least one non-transitory computer-readable storage medium storing processor-executable instruction that, when executed by at least one processor, cause the at least one processor to perform a method for selecting at least one embryo for transfer to a subject in furtherance of an in vitro fertilization (IVF) treatment, the method comprising:
obtaining video data for a plurality of embryos including a first embryo, the video data comprising a first sequence of image frames depicting the first embryo;
obtaining electronic health data for the subject, the electronic health data comprising information about the IVF treatment;
predicting, using the video data and the electronic health data, respective degrees of viability of at least some of the plurality of embryos, the predicting comprising:
processing the electronic health data and the first sequence of image frames using at least one trained machine learning model to obtain a first degree of viability of the first embryo; and
selecting, from among the at least some of the plurality of embryos and based on the predicted degrees of viability including the first degree of viability, the at least one embryo for transfer to the subject.
